(19) United States
(12) Patent Application Publication
(10) Pub. No.: US 2012/0076262 A1
(43) Pub. Date: Mar. 29, 2012

(54) RADIOLOGICAL IMAGE DISPLAYING DEVICE AND METHOD

(75) Inventor: Takashi TAJIMA, Kanagawa (JP)

(73) Assignee: FUJIFILM CORPORATION, Tokyo (JP)

(21) Appl. No.: 13/247,503

(22) Filed: Sep. 28, 2011

(30) Foreign Application Priority Data
Sep. 29, 2010 (JP) 2010-219121

(51) Int. Cl. A61B 6/02

(52) U.S. Cl. 378/41

(57) ABSTRACT

The stereoscopic effect of auxiliary lines for making a stereoscopic effect easy to figure out is matched with the stereoscopic effect of a subject included in a stereoscopic image when displaying a stereoscopic image using a radiological image. Auxiliary lines are added to two radiological images for displaying a stereoscopic image. The auxiliary lines are formed by arraying a plurality of grids so as to be viewed in the depth direction. The auxiliary lines are generated according to the compression thickness when compressing the subject to radiograph the subject, the radiographing technique, and the compression pressure when compressing the subject to radiograph the subject.

### BACKGROUND OF THE INVENTION

1. Field of the Invention

The present invention relates to a radiological image displaying device and method for displaying a stereoscopic image of a subject.

2. Description of the Related Art

In the related art, realizing stereoscopic viewing using parallax by displaying a combination of a plurality of images is known. Such an image which can be stereoscopically viewed (hereinafter, referred to as a stereoscopic image or a three-dimensional image) is displayed based on a plurality of images with parallax acquired by radiographing the same subject from different directions.

Various methods for making it easy to figure out the stereoscopic effect of structures included in an image when a stereoscopic image is displayed have been proposed. For example, according to a technique disclosed in JP2006-271739A, corresponding points in two eye-fundus images acquired by photographing the eye fundus from different direction, for displaying an eye-fundus image as a stereoscopic image, and contour lines representing the same depth of the eye fundus are added on the two eye-fundus images based on the corresponding points. By displaying a stereoscopic image using the eye-fundus images on which the contour lines are added in this way, the sense of depth of the eye fundus can be made easy to figure out. Moreover, according to the technique disclosed in JP2006-271739A, the colors of the contour lines are different in accordance with the depth so as to make it easy to figure out the difference in the sense of depth of the eye fundus.

In addition, a method of displaying a grid line showing the depth direction when displaying an image to be measured in a three-dimensional manner has been proposed as a method of making easy to figure out a sense of depth of a stereoscopic image (refer to JP2009-053147A).

On the other hand, generation of such a stereoscopic image is used not only in the field of digital cameras, televisions but also in the field of radiography. That is, a technique which involves irradiating radioactive rays onto a subject from different radiographing directions, detecting radioactive rays having passed through the subject by a radiation detector to acquire a plurality of radiological images having parallaxes, and displaying a stereoscopic image using these radiological images has been performed. By using such a stereoscopic image, an observer can observe radiological images having a sense of depth and more easily make a diagnosis.

### SUMMARY OF THE INVENTION

However, since the radiological image is a transfer image of the inside of a subject, structures inside the subject, such as bones, various tissues and tumor masses, or lesions, such as calcification, are included in an overlapped state. Thus, when a stereoscopic image is displayed using radiological images, since structures are displayed stereoscopically with a stereoscopic effect so as to stay afloat in a space, it is difficult to catch the sense of depth of structures in the stereoscopic radiological image. Moreover, a technique of using a 3D cursor capable of moving in the depth direction as well as in a planar direction to input necessary instructions on a displayed stereoscopic image may be considered. However, it is difficult to match the sense of depth of the 3D cursor so as to be identical to the sense of depth of a region of interest such as a lesion in a stereoscopic image. For this reason, when displaying a stereoscopic image using a radiological image, it is preferable to display auxiliary lines, which are formed by arraying a plurality of mesh-like grids with different senses of depth, so as to match the stereoscopic effect of a subject included in the stereoscopic image. In this case, in order to display the auxiliary lines, it may be considered to use the methods disclosed in JP2006-271739A and JP2009-053147A.

In the method disclosed in JP2006-271739A, it is necessary to calculate corresponding points between two images in order to display the contour with the same sense of depth. However, since a plurality of structures overlap in the depth direction in the radiological image, it is very difficult to calculate the corresponding points with the same sense of depth. For this reason, it is difficult to match the stereoscopic effect of auxiliary lines with the stereoscopic effect of a subject included in a stereoscopic image by applying the method disclosed in JP2006-271739A, in which the contour showing the same depth is used, to the stereoscopic image of the radiological image. In addition, in the method disclosed in JP2009-053147A, grid lines are displayed at the position, which is set in advance, on an image in order to measure the stereoscopic effect. Accordingly, even if the method disclosed in JP2009-053147A is added to the stereoscopic image of the radiological image, the stereoscopic effect between the frontmost side and the rearmost side in the displayed grids does not match the stereoscopic effect of a subject included in the stereoscopic image.

In view of the above, it is an object of the present invention to match the stereoscopic effect of auxiliary lines, which are for making easy to figure out a sense of depth, with the stereoscopic effect of a subject included in a stereoscopic image when displaying a stereoscopic image using a radiological image.

The present invention has been made in view of the above-mentioned problems and an object of the present invention is to provide a radiological image displaying device including: an image acquisition unit that acquires a plurality of radiological images for displaying a stereoscopic image of a subject; a display control unit that displays the stereoscopic image on a display unit using the plurality of radiological images; an auxiliary line generating unit that generates auxiliary lines, which are formed by arraying a plurality of grids so as to be viewed in a depth direction, according to the compression thickness when compressing the subject to radiograph the subject; and an auxiliary line adding unit that adds the auxiliary lines to the plurality of radiological images so as to be able to be stereoscopically viewed.

Any kind of shape, such as a rectangle, a circle, and a triangle, may be used as the shape of a grid, a rectangle is preferable. In addition, each grid is divided into a plurality of regions in the shape of a mesh.

In addition, a breast may be mentioned as an example of the subject compressed and radiographed.

In addition, in the radiological image displaying device according to the aspect of the present invention, the auxiliary line generating unit may be a unit that generates the auxiliary lines by disposing the plurality of grids so as to be included in a range of the compression thickness.

In this case, the auxiliary line generating unit may be a unit that generates the auxiliary lines such that grids are disposed at positions of a frontmost structure and a rearmost structure included in the stereoscopic image and at least one grid is disposed at a position dividing the grids equally when displaying the stereoscopic image using the plurality of radiological images to which the auxiliary lines are added.

In addition, in the radiological image displaying device according to the aspect of the present invention, the auxiliary line generating unit may be a unit that also generates the auxiliary lines according to the shape of the subject included in the radiological image.

In this case, the auxiliary line generating unit may be a unit that generates the auxiliary lines by setting the size of each grid in a direction perpendicular to the depth direction so as to include a range of the shape of the subject.

In addition, in the radiological image displaying device according to the aspect of the present invention, the auxiliary line generating unit may be a unit that estimates the shape of the subject based on the compression thickness, compression pressure when radiographing the subject with compressing the subject, and/or a radiographing technique when radiographing the subject.

In addition, in the radiological image displaying device according to the aspect of the present invention, the auxiliary line generating unit may be a unit that estimates the shape of the subject based on a region of the subject included in the radiological image.

According to another aspect of the present invention, a radiological image displaying method in a radiological image displaying device including an image acquisition unit that acquires a plurality of radiological images for displaying a stereoscopic image of a subject and a display control unit that displays the stereoscopic image on a display unit using the plurality of radiological images includes: generating auxiliary lines, which are formed by arraying a plurality of grids so as to be viewed in a depth direction, according to the compression thickness when compressing the subject to radiograph the subject; and adding the auxiliary lines to the plurality of radiological images so as to be able to be stereoscopically viewed.

According to the aspect of the present invention, when adding the auxiliary lines, which are formed by arraying the plurality of grids so as to be viewed in the depth direction, to the plurality of radiological images so as to be able to be stereoscopically viewed, the auxiliary lines are generated according to the compression thickness when compressing the subject to radiograph the subject. Accordingly, the auxiliary lines can be generated so that the stereoscopic effect of the auxiliary lines matches the stereoscopic effect of the subject included in the stereoscopic image. As a result, by using the auxiliary lines when making a diagnosis using the stereoscopic image, the stereoscopic effect of the structure included in the subject which is stereoscopically viewed can be easily figured out.

In addition, by generating the auxiliary lines according to the compression thickness when compressing the subject to radiograph the subject, it is possible to match the stereoscopic effect of the auxiliary lines with the stereoscopic effect of the subject included in the stereoscopic image when displaying a stereoscopic image using a radiological image acquired by compressing a subject, such as a breast, to radiograph the subject.

In addition, also by generating the auxiliary lines according to the shape of the subject included in the radiological image, the auxiliary lines can be generated so as to include the range of the shape of the subject. As a result, by using the auxiliary lines when making a diagnosis using the stereoscopic image, the stereoscopic effect of the structures included within the entire subject which is stereoscopically viewed can be easily figured out.

### BRIEF DESCRIPTION OF THE DRAWINGS

FIG. 1 is a view showing the schematic configuration of a radiological image radiographing apparatus to which a radiological image displaying device according to an embodiment of the present invention is added.

FIG. 2 is a view when an arm unit of the radiological image radiographing apparatus shown in FIG. 1 is seen from the right side of FIG. 1.

FIG. 3 is a block diagram showing the schematic configuration inside a computer of the radiological image radiographing apparatus shown in FIG. 1.

FIG. 4 is a view showing an auxiliary line.

FIG. 5 is a view showing the compression state of a breast at the time of radiographing.

FIG. 6 is a view for explaining the arrangement of grids.

FIG. 7 is a view for explaining the arrangement of grids.

FIG. 8 is a view showing a radiological image depending on the compression thickness and radiographing technique.

FIG. 9 is a view showing a grid with a size depending on the compression thickness and radiographing technique.

FIG. 10 is a view showing a radiological image depending on the compression thickness and the compression pressure.

FIG. 11 is a view showing a grid with a size depending on the compression thickness and the compression pressure.

FIG. 12 is a view showing a radiological image to which auxiliary lines are added.

FIG. 13 is a flow chart showing the processing performed in the present embodiment.

### DESCRIPTION OF THE PREFERRED EMBODIMENTS

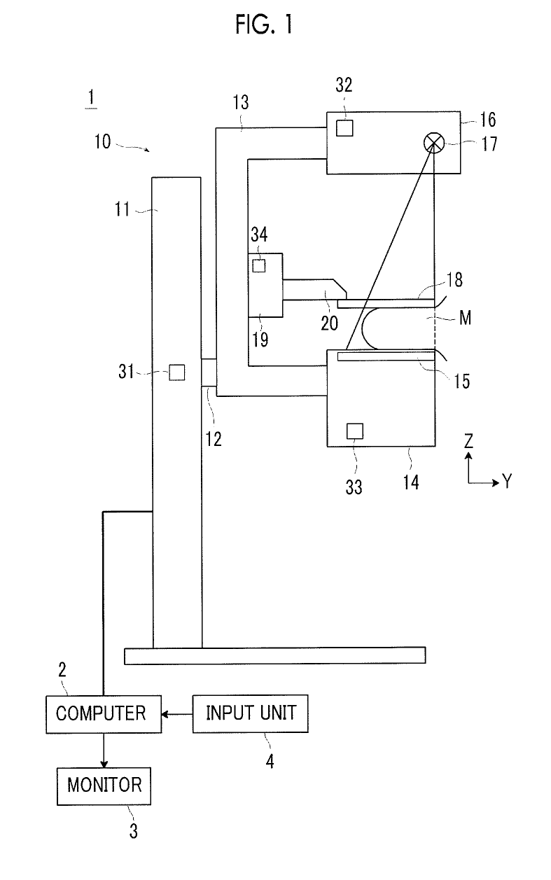

Hereinafter, embodiments of the present invention will be described with reference to the accompanying drawings. FIG. 1 is a view showing the schematic configuration of a radiological image radiographing apparatus to which a radiological image displaying device according to an embodiment of the present invention is added. A radiological image radiographing apparatus 1 according to the present embodiment acquires a plurality of radiological images by radiographing a breast M from different radiographing directions in order to generate a stereoscopic image for stereoscopic viewing of a radiological image of the breast M. As shown in FIG. 1, the radiological image radiographing apparatus 1 includes a radiographing unit 10 which includes an image acquisition unit (not shown), a computer 2 connected to the radiographing unit 10, and a monitor 3 (display unit) and an input unit 4 connected to the computer 2.

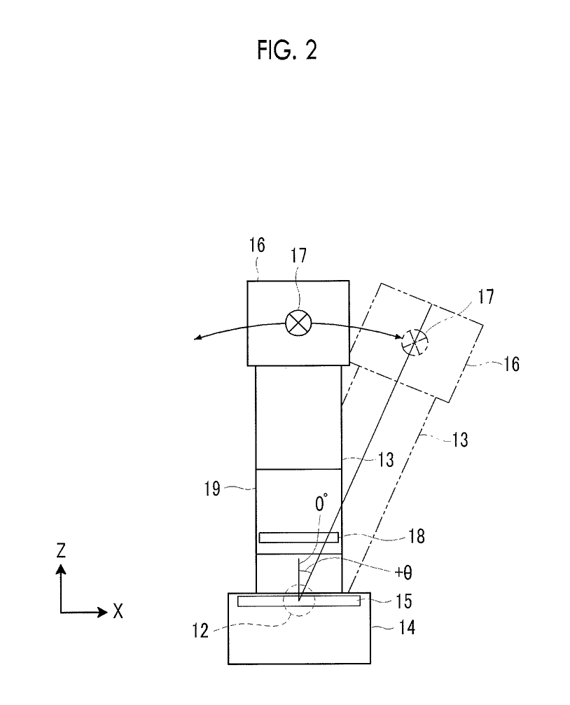

The radiographing unit 10 includes a pedestal 11, a rotary shaft 12 which can rotate and move up and down (in the Z direction) with respect to the pedestal 11, and an arm unit 13 connected to the pedestal 11 by the rotary shaft 12. In addition, FIG. 2 shows the arm unit 13 when viewed from the right side in FIG. 1.

The arm unit 13 has a shape of a letter C. A radiation plane 14 is fixed to one end of the arm unit 13, and an irradiating unit 16 is fixed to the other end so as to face the radiation plane 14. Rotation and up-and-down movement of the arm unit 13 are controlled by an arm controller 31 provided in the pedestal 11.

A radiation detector 15, such as a flat panel detector, and a detector controller 33 which controls reading of a charge signal from the radiation detector 15 are provided inside the radiation plane 14.

In addition, a circuit board on which a charge amplifier that converts a charge signal read from the radiation detector 15 into a voltage signal, a correlated double sampling circuit that samples a voltage signal output from the charge amplifier, an AD converter that converts a voltage signal into a digital signal, and the like are provided is placed inside the radiation plane 14.

In addition, the radiation plane 14 is configured to be able to rotate with respect to the arm unit 13. Accordingly, even when the arm unit 13 rotates with respect to the pedestal 11, the direction of the radiation plane 14 can be fixed with respect to the pedestal 11.

The radiation detector 15 can perform recording and reading of a radiological image repeatedly. A so-called direct-conversion radiation detector which generates an electric charge by direct reception of radiation may be used, or a so-called indirect-conversion radiation detector which converts radiation into visible light and then converts the visible light into a charge signal may be used. Moreover, as a method of reading a radiological image signal, a so-called TFT reading method in which a radiological image signal is read by ON/OFF of a TFT (thin film transistor) switch or a so-called optical reading method in which a radiological image signal is read by irradiation of reading light is preferably used. However, other methods may be used without being limited to the above methods.

A radiation source 17 and a radiation source controller 32 are provided in the irradiating unit 16. The radiation source controller 32 controls an irradiation timing of radiation from the radiation source 17 and the radiation generating conditions (tube current, time, tube current time product, and the like) in the radiation source 17.

In addition, a compression plate 18 provided above the radiation plane 14 to compress a breast, a supporting unit 20 which supports the compression plate 18, and a moving mechanism 19 which moves the supporting unit 20 up and down (in the Z direction) are provided in the middle of the arm unit 13. The position and the pressure of the compression plate 18 are controlled by a compression plate controller 34. Here, the position of the compression plate 18 is a position with a state, in which the compression plate 18 is in close contact with the radiation plane 14, as a reference. Accordingly, the position of the compression plate 18 indicates a distance between the detection plane of the radiation plane 14 and the bottom surface of the compression plate 18 when compressing the breast M, that is, a compression thickness which is the thickness of the compressed breast M. In addition, the compression plate controller 34 outputs the information regarding the position and the pressure of the compression plate 18 to the computer 2.

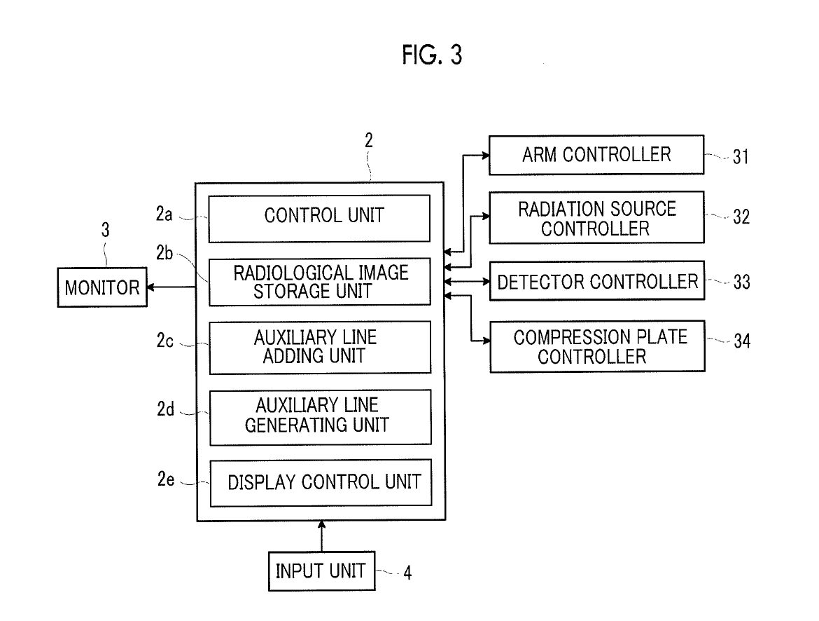

The computer 2 includes a central processing unit (CPU) and a storage device, such as a semiconductor memory, a hard disk, or an SSD. By such hardware, a control unit 2a, a radiological image storage unit 2b, an auxiliary line adding unit 2c, an auxiliary line generating unit 2d, and a display control unit 2e shown in FIG. 3 are formed.

The control unit 2a outputs predetermined control signals to various kinds of controllers 31 to 34 to control the entire apparatus.

The radiological image storage unit 2b stores two radiological images (referred to as G1 and G2) detected by the radiation detector 15 after radiographing from two different radiographing directions.

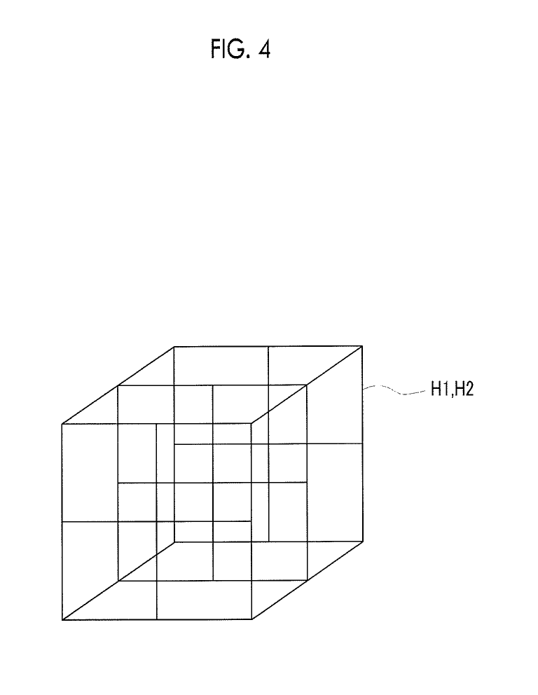

The auxiliary line adding unit 2c adds auxiliary lines H1 and H2 for expressing a sense of depth of a stereoscopic image to two radiological images G1 and G2 so as to be able to be stereoscopically viewed when displaying a stereoscopic image using the two radiological images G1 and G2 on the monitor 3. FIG. 4 is a view showing auxiliary lines. As shown in FIG. 4, auxiliary lines are formed by arraying a plurality of rectangular grids in the depth direction according to the stereoscopic effect of a stereoscopic image. Each grid is divided into a plurality of regions in the shape of a mesh. Moreover, in FIG. 4, the auxiliary line between the grids extending in the depth direction is not an essential element but is provided in order to make the present invention understood more easily. However, since an auxiliary line is easily viewed by providing the auxiliary line in the depth direction, a more effective stereoscopic effect can be achieved.

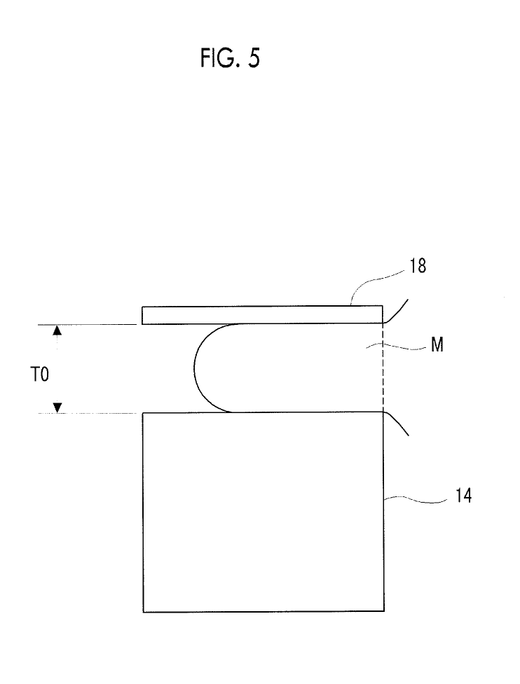

The auxiliary line generating unit 2d generates the auxiliary lines H1 and H2. In this case, the auxiliary line generating unit 2d uses the information regarding the position of the compression plate 18, which is output from the compression plate controller 34, as the thickness of the breast M (compression thickness) in a state compressed by the compression plate 18 at the time of radiographing. FIG. 5 is a view showing the compression state of the breast M at the time of radiographing. As shown in FIG. 5, the breast M is compressed on the radiation plane 14 by the compression plate 18. In this case, the thickness of the breast M, that is, the compression thickness is T0. Therefore, the auxiliary line generating unit 2d generates the auxiliary lines H1 and H2 by arraying the frontmost grid and the rearmost grid such that the frontmost grid of the plurality of grids, which form the auxiliary lines H1 and H2, is stereoscopically viewed at the position of the surface of the compressed breast M facing the radiation source (that is, the surface adjacent to the compression plate 18) and the rearmost grid is stereoscopically viewed at the position of the surface of the compressed breast M facing the radiation plane 14 (that is, the surface adjacent to the radiation plane 14). That is, it is possible to generate auxiliary lines depending on the compression thickness.

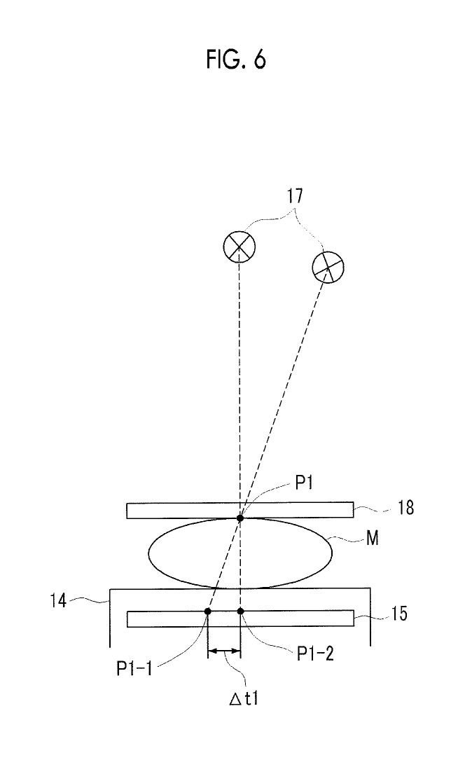

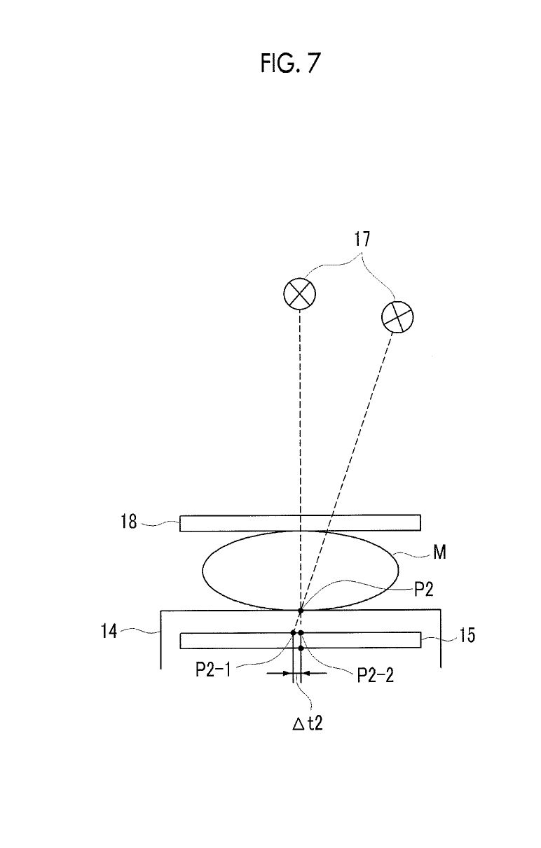

FIGS. 6 and 7 are views for explaining the arrangement of grids. As shown in FIG. 6, a point P1 of the compressed breast M which is closest to the radiation source is projected onto points P1-1 and P1-2 of the detector 15 by radiation emitted from the radiation sources 17 located in two different directions. A position difference Δt1 between the points P1-1 and P1-2 becomes the parallax of the point P1 on the radiological images G1 and G2.

On the other hand, as shown in FIG. 7, a point P2 of the compressed breast M which is closest to the radiation plane is projected onto points P2-1 and P2-2 of the detector 15 by radiation emitted from the radiation sources 17 located in two different directions. A position difference Δt2 between the points P2-1 and P2-2 becomes the parallax of the point P2 on the radiological images G1 and G2. In addition, as shown in FIGS. 6 and 7, the parallax Δt1 is larger than the parallax Δt2. Accordingly, when displaying a stereoscopic image using the radiological images G1 and G2, the stereoscopic effect of the breast M based on radiation at the radiation source side is larger.

The auxiliary line generating unit 2d generates the auxiliary lines H1 and H2 such that parallax of the frontmost grid in the auxiliary lines H1 and H2 becomes Δt1 and parallax of the rearmost grid becomes Δt2 when adding the auxiliary lines H1 and H2 to the radiological images G1 and G2. Here, the parallax Δt1 and Δt2 can be geometrically calculated based on the radiation source distance which is a distance from the detection plane of the detector 15 to the radiation source 17, the angle of convergence, and the compression thickness. In addition, a grid between the frontmost grid and the rearmost grid is disposed so as to show parallax which divides the parallax Δt1 and Δt2 equally, thereby generating the auxiliary lines H1 and H2. In addition, the number of grids may be set in advance or may be set to increase according to an increase in the compression thickness.

In addition, the auxiliary line generating unit 2d predicts the shape of the breast M at the time of radiographing using not only the compression thickness but also the information regarding the radiographing technique at the time of radiographing or the pressure of the compression plate and sets the sizes of grids, which form the auxiliary lines H1 and H2, according to the predicted shape. Here, examples of the technique of radiographing the breast include cranio-caudal (CC) radiographing in which radiographing is performed by emitting radiation from above, medial-lateral (ML) radiographing in which radiographing is performed by emitting radiation from the side, and medio-lateral oblique (MLO) radiographing in which radiographing is performed by emitting radiation from an oblique direction. In addition, these radiographing techniques are input through the input unit 4 by the operator at the time of radiographing.

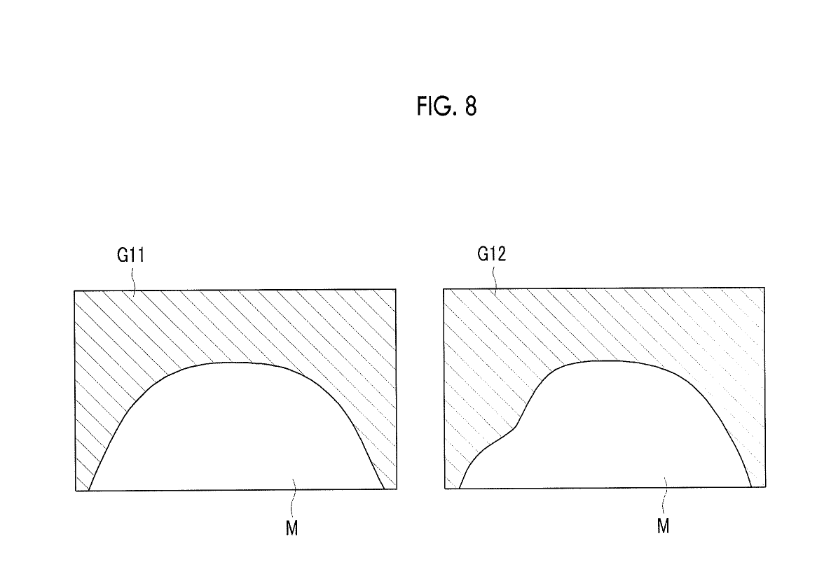

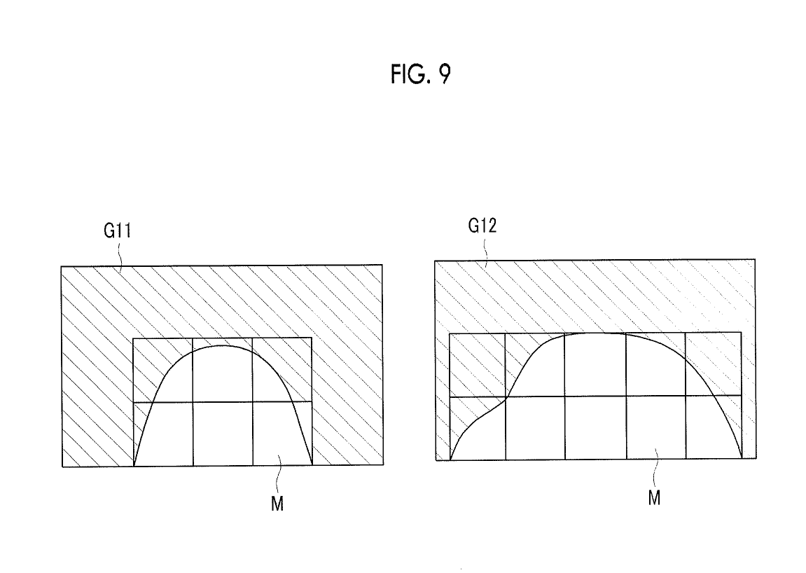

Here, in the CC radiographing and the ML radiographing, only the breast M can be included in a radiological image as shown in a radiological image G11 of FIG. 8. In the MLO radiographing, however, the armpit of the subject tends to be included in a radiological image as shown in a radiological image G12. In addition, the compression thickness is large in the case of the relatively large breast M, and the compression thickness is small in the case of the relatively small breast M. For this reason, the auxiliary line generating unit 2d estimates the shape of the breast M from the information regarding the radiographing technique and the information regarding the compression thickness. In addition, the size of a grid is set so as to include a range of the estimated shape of the breast M. Specifically, as shown in FIG. 9, the size of a grid is set so as to include a range of the shape of the breast M for each of the radiological images G11 and G12. In addition, it is preferable that the number of divisions for making a grid in the shape of a mesh be set according to the size of the grid.

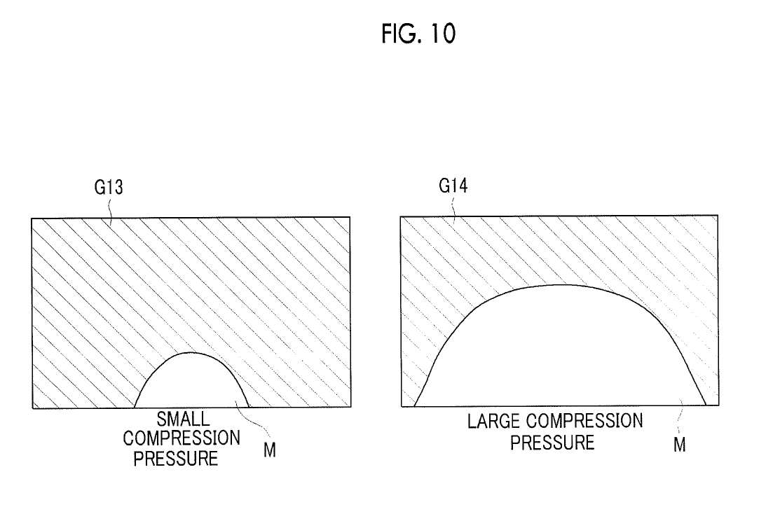

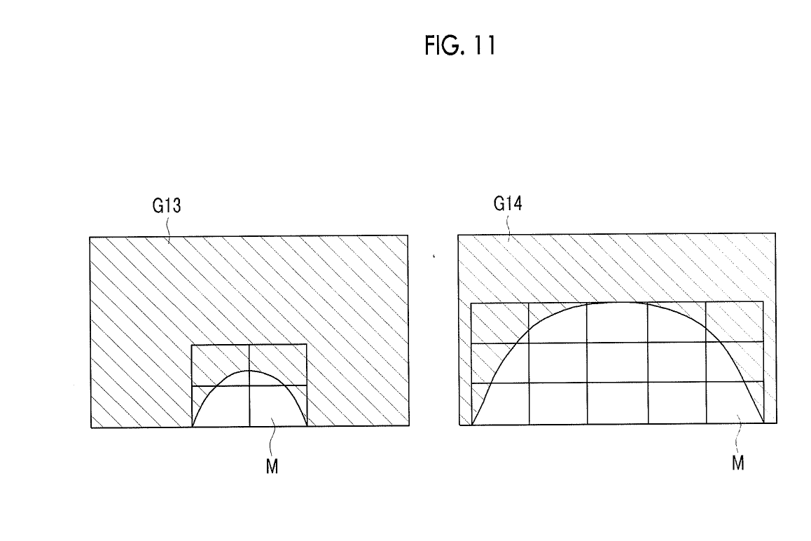

On the other hand, as shown in a radiological image G13 of FIG. 10, the compression thickness of the relatively small breast M decreases as the pressure increases. However, as shown in a radiological image G14, the compression thickness of the relatively large breast M is large even if the pressure is large. For this reason, the auxiliary line generating unit 2d estimates the shape of the breast M from the information regarding the pressure and the compression thickness. In addition, the size of a grid is set so as to include the breast with the estimated shape. Specifically, as shown in FIG. 11, the size of a grid is set so as to include a range of the shape of the breast M for each of the radiological images G13 and G14. As a result, it is also possible to generate auxiliary lines according to the shape of the subject.

In addition, the auxiliary line adding unit 2c may recognize the shape of the breast M included in each of the radiological images G1 and G2 by performing processing, such as edge recognition, on the radiological images G1 and G2 and set the size of a grid based on the recognition result so as to include the breast with the recognized shape.

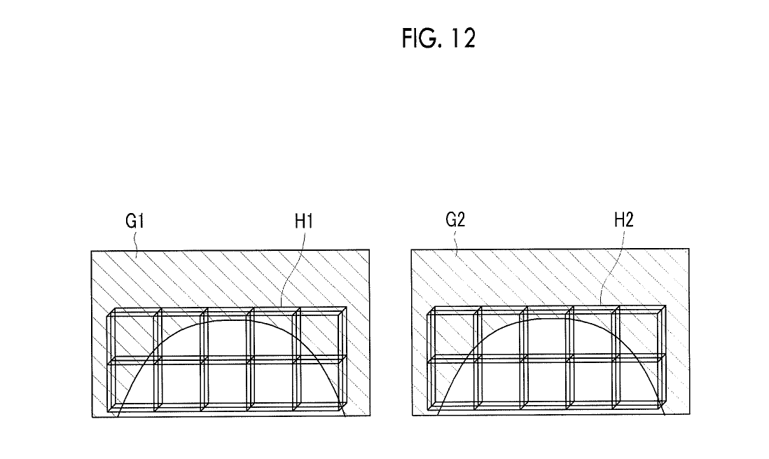

The auxiliary line adding unit 2c adds the auxiliary lines H1 and H2 generated by the auxiliary line generating unit 2d to the radiological images G1 and G2, respectively. FIG. 12 is a view showing two radiological images to which auxiliary lines are added. As shown in FIG. 12, the auxiliary lines H1 and H2 formed by arraying grids are added to the radiological images G1 and G2 so as to overlap the breast M in the radiological images G1 and G2, respectively. In addition, in FIG. 12, each of the auxiliary lines H1 and H2 is formed by three rectangular grids, and each grid is divided into a plurality of regions (here, six regions) in the shape of a mesh. In addition, the positional difference, that is, parallax of each grid on the radiological images G1 and G2 decreases from the front side toward the rear side. For this reason, when displaying a stereoscopic image using the radiological images G1 and G2 to which the auxiliary lines H1 and H2 are added, the stereoscopic image is viewed in a state where three grids are arrayed from the front side to the rear side.

In addition, when displaying a stereoscopic image using the radiological images G1 and G2 to which the auxiliary lines H1 and H2 are added, parallax of the auxiliary lines H1 and H2 may be changed by an input from the input unit 4. In this case, the stereoscopic effect of the auxiliary lines can be combined with the stereoscopic effect of the breast M included in the stereoscopic image. In addition, whether to display the auxiliary lines H1 and H2 may be changed by an input from the input unit 4. In addition, since the brightness of a region of the breast M in the radiological images G1 and G2 is relatively high, it is preferable that the brightness of each of the auxiliary lines H1 and H2 be low.

The display control unit 2e performs predetermined processing on the radiological images G1 and G2 to which the auxiliary lines H1 and H2 are added and then displays a stereoscopic image of the breast M on the monitor 3.

The monitor 3 is configured to be able to perform three-dimensional display of a stereoscopic image using the two radiological images G1 and G2 output from the computer 2. As an example of the three-dimensional display method of the monitor 3, a method may be adopted in which two radiological images are displayed using two screens and one of the radiological images is incident on the right eye of an observer and the other radiological image is incident on the left eye of the observer using a half mirror, a polarization glass, and the like to thereby display a stereoscopic image. In addition, a method of displaying a stereoscopic image by superimposing two radiological images and making these radiological images observable with a polarization glass may also be used. In addition, the monitor 3 may be formed by a 3D display, and a method by which stereoscopic viewing of two radiological images is possible, such as a parallax barrier method and a lenticular method, may be used.

The input unit 4 includes a keyboard or a pointing device, such as a mouse, and receives from an operator an input of radiographing conditions, an input of a radiographing start instruction, and the like.

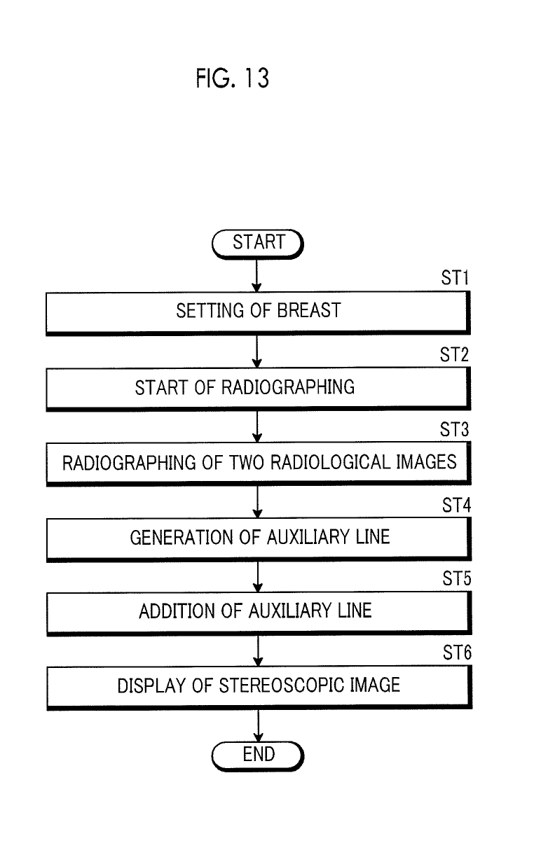

Next, processing performed in the present embodiment will be described. FIG. 13 is a flow chart showing the processing performed in the present embodiment. First, the breast M of a patient is placed on the radiation plane 14 and is compressed with predetermined pressure by the compression plate 18 (step ST1). Then, various radiographing conditions are input through the input unit 4 and then an instruction to start radiographing is input (step ST2).

If there is an instruction to start radiographing through the input unit 4, radiographing of two radiological images for displaying the stereoscopic image of the breast M is performed (step ST3). Specifically, first, the control unit 2a reads an angle of convergence θ stored and outputs the information regarding the read angle of convergence θ to the arm controller 31. In addition, the arm controller 31 receives the information regarding the angle of convergence θ output from the control unit 2a and outputs a control signal to make the arm unit 13 positioned in a direction perpendicular to the radiation plane 14 (direction of 0°) as shown by the solid line in FIG. 2.

Then, in a state where the arm unit 13 is positioned perpendicular to the radiation plane 14 according to the control signal output from the arm controller 31, the control unit 2a outputs control signals to the radiation source controller 32 and the detector controller 33 in order to perform irradiation and reading of a radiological image signal. In addition, the position of the radiation source 17 in this state becomes a viewpoint as a reference. According to this control signal, radiation is emitted from the radiation source 17, a radiological image obtained by radiographing the breast M from the direction of 0° is detected by the radiation detector 15, a radiological image signal is read from the radiation detector 15 by the detector controller 33, and predetermined signal processing is performed on the radiological image signal. Then, the result is stored as the radiological image G1 as a reference in the radiological image storage unit 2b of the computer 2.

Then, the arm controller 31 outputs a control signal to make the arm unit 13 rotate by +θ° in a direction perpendicular to the radiation plane 14 as shown by the virtual line in FIG. 2. Then, in a state where the arm unit 13 has rotated by +θ° according to the control signal output from the arm controller 31, the control unit 2a outputs control signals to the radiation source controller 32 and the detector controller 33 in order to perform irradiation and reading of a radiological image signal. According to this control signal, radiation is emitted from the radiation source 17, a radiological image obtained by radiographing the breast M from the direction of +θ° is detected by the radiation detector 15, a radiological image signal is read by the detector controller 33, and predetermined signal processing is performed. Then, the result is stored as the radiological image G2 in the radiological image storage unit 2b of the computer 2.

Then, the two radiological images G1 and G2 stored in the radiological image storage unit 2b are read, and the auxiliary lines H1 and H2 added to the radiological images G1 and G2 are generated by the auxiliary line generating unit 2d (step ST4). Then, the auxiliary line adding unit 2c adds the auxiliary lines H1 and H2 to the radiological images G1 and G2, respectively (step ST5). Then, predetermined processing is performed on radiological images GS1 and GS2, to which the auxiliary lines H1 and H2 are added, by the display control unit 2e. Then, the result is output to the monitor 3, and a stereoscopic image of the breast M is displayed on the monitor 3 (step ST6).

Thus, in the present embodiment, when adding the auxiliary lines H1 and H2, which are formed by arraying a plurality of grids so as to be viewed in the depth direction, to the plurality of radiological images G1 and G2 so that they can be stereoscopically viewed according to a sense of depth of a stereoscopic image, the auxiliary lines H1 and H2 are generated according to the thickness of the breast M. For this reason, the auxiliary lines H1 and H2 can be generated so that the stereoscopic effect of the auxiliary lines H1 and H2 matches the stereoscopic effect of the breast M included in the stereoscopic image. As a result, by using the auxiliary lines H1 and H2 when making a diagnosis using the stereoscopic image, the stereoscopic effect of the structure included in the breast M which is stereoscopically viewed can be easily figured out.

In addition, by setting the sizes of the auxiliary lines H1 and H2 according to the shape of the breast M included in the radiological images G1 and G2, the auxiliary lines H1 and H2 can be generated so as to include the entire shape of the breast M included in the radiological images G1 and G2. As a result, by using the auxiliary lines when making a diagnosis using the stereoscopic image, the stereoscopic effect of the structure included in the entire breast M which is stereoscopically viewed can be easily figured out.

Moreover, in the embodiment described above, auxiliary lines formed by arraying three grids in the depth direction are generated. However, it is also possible to use an arbitrary number according to the stereoscopic effect of a stereoscopic image as long as the number of grids is 2 or more. In addition, the arbitrary number of divisions may also be used as the number of divisions of mesh-like regions in a grid.

In addition, although the rectangular grid is used in the embodiment described above, it is also possible to use a grid with any kind of shape, such as a circle or a triangle.

In addition, although a radiological image acquired by radiographing the breast M from the direction of 0° is used as the radiological image G1 as a reference in the embodiment described above, an image acquired by radiographing the breast M from a different direction from 0° may be used as a reference of two radiological images for displaying a stereoscopic image. In this case, a stereoscopic image is preferably displayed using a radiological image radiographed from the different direction from 0° as the radiological image G1 as a reference.

In addition, although the radiological image radiographing apparatus to which the radiological image displaying device according to the above-described embodiment of the present invention is added is used as an apparatus which radiographs a radiological image of a breast, the subject is not limited to a breast. For example, a radiological image radiographing apparatus which radiographs a chest, a head, and the like may also be used. In this case, parts, such as the chest or the head, are not compressed at the time of radiographing unlike the breast. Therefore, when radiographing these parts, it is preferable to measure the thickness of the corresponding part and to generate an auxiliary line using the measured thickness.

What is claimed is:

1. A radiological image displaying device comprising:
an image acquisition unit that acquires a plurality of radiological images for displaying a stereoscopic image of a subject;
a display control unit that displays the stereoscopic image on a display unit using the plurality of radiological images;
an auxiliary line generating unit that generates auxiliary lines, which are formed by arraying a plurality of grids so as to be viewed in a depth direction, according to a compression thickness when compressing the subject to radiograph the subject; and
an auxiliary line adding unit that adds the auxiliary lines to the plurality of radiological images so as to be able to be stereoscopically viewed.

2. The radiological image displaying device according to claim 1,
wherein the auxiliary line generating unit is a unit that generates the auxiliary lines by disposing the plurality of grids so as to be included in a range of the compression thickness.

3. The radiological image displaying device according to claim 2,
wherein the auxiliary line generating unit is a unit that generates the auxiliary lines such that grids are disposed at positions of a frontmost structure and a rearmost structure included in the stereoscopic image and at least one grid is disposed at a position dividing the grids equally when displaying the stereoscopic image using the plurality of radiological images to which the auxiliary lines are added.

4. The radiological image displaying device according to claim 1,
wherein the auxiliary line generating unit is a unit that also generates the auxiliary lines according to the shape of the subject included in the radiological image.

5. The radiological image displaying device according to claim 2,
wherein the auxiliary line generating unit is a unit that also generates the auxiliary lines according to the shape of the subject included in the radiological image.

6. The radiological image displaying device according to claim 3,
wherein the auxiliary line generating unit is a unit that also generates the auxiliary lines according to the shape of the subject included in the radiological image.

7. The radiological image displaying device according to claim 4,
wherein the auxiliary line generating unit is a unit that generates the auxiliary lines by setting the size of each grid in a direction perpendicular to the depth direction so as to include a range of the shape of the subject.

8. The radiological image displaying device according to claim 4,
wherein the auxiliary line generating unit is a unit that estimates the shape of the subject based on the compression thickness, compression pressure when compressing the subject to radiograph the subject, and/or a radiographing technique when radiographing the subject.

9. The radiological image displaying device according to claim 5,
wherein the auxiliary line generating unit is a unit that estimates the shape of the subject based on the compression thickness, compression pressure when radiographing the subject with compressing the subject, and/or a radiographing technique when radiographing the subject.

10. The radiological image displaying device according to claim 6,
wherein the auxiliary line generating unit is a unit that estimates the shape of the subject based on the compression thickness, compression pressure when radiographing the subject with compressing the subject, and/or a radiographing technique when radiographing the subject.

11. The radiological image displaying device according to claim 7,
wherein the auxiliary line generating unit is a unit that estimates the shape of the subject based on the compression thickness, compression pressure when radiographing the subject with compressing the subject, and/or a radiographing technique when radiographing the subject.

12. The radiological image displaying device according to claim 4,
wherein the auxiliary line generating unit is a unit that estimates the shape of the subject based on a region of the subject included in the radiological image.

13. The radiological image displaying device according to claim 5,
wherein the auxiliary line generating unit is a unit that estimates the shape of the subject based on a region of the subject included in the radiological image.

14. The radiological image displaying device according to claim 6,
wherein the auxiliary line generating unit is a unit that estimates the shape of the subject based on a region of the subject included in the radiological image.

15. The radiological image displaying device according to claim 7,
wherein the auxiliary line generating unit is a unit that estimates the shape of the subject based on a region of the subject included in the radiological image.

16. A radiological image display method in a radiological image displaying device including an image acquisition unit that acquires a plurality of radiological images for displaying a stereoscopic image of a subject and a display control unit that displays the stereoscopic image on a display unit using the plurality of radiological images, the method comprising:
generating auxiliary lines, which are formed by arraying a plurality of grids so as to be viewed in a depth direction, according to a compression thickness when compressing the subject to radiograph the subject; and
adding the auxiliary lines to the plurality of radiological images so as to be able to be stereoscopically viewed.
